SEQUENCE LISTING (17 sequences)

SEQ ID NO: 1 - protein, 29 aa, Homo sapiens
HSQGTFTSDYSKYLDSRRAQDFVQWLMNT

SEQ ID NO: 2 - protein, 4 aa, Artificial Sequence
  Synthetic Sequence - N-terminal peptide (1-4)
HSQG

SEQ ID NO: 3 - protein, 25 aa, Artificial Sequence
  Synthetic sequence - C-terminal peptide (5-29)
TFTSDYSKYLDSRRAQDFVQWLMNT

SEQ ID NO: 4 - protein, 25 aa, Artificial Sequence
  Synthetic Sequence - C-terminal peptide with pseudoproline pDS
  Xaa (11)..(12): Asp-Ser pseudoproline dipeptide
TFTSDYSKYLXXRRAQDFVQWLMNT

SEQ ID NO: 5 - protein, 25 aa, Artificial Sequence
  Synthetic Sequence - C-terminal peptide with pseudoproline pYS
  Xaa (6)..(7): Tyr-Ser pseudoproline dipeptide
TFTSDXXKYLDSRRAQDFVQWLMNT

SEQ ID NO: 6 - protein, 25 aa, Artificial Sequence
  Synthetic Sequence - C-terminal peptide with pseudoproline pYS and pDS
  Xaa (6)..(7): Tyr-Ser pseudoproline dipeptide
  Xaa (11)..(12): Asp-Ser pseudoproline dipeptide
TFTSDXXKYLXXRRAQDFVQWLMNT

SEQ ID NO: 7 - protein, 25 aa, Artificial Sequence
  Synthetic Sequence - C-terminal peptide with pseudoproline pTS
  Xaa (3)..(4): Thr-Ser pseudoproline dipeptide
TFXXDYSKYLDSRRAQDFVQWLMNT

SEQ ID NO: 8 - protein, 25 aa, Artificial Sequence
  Synthetic Sequence - C-terminal peptide with pseudoproline pTS and pDS
  Xaa (3)..(4): Thr-Ser pseudoproline dipeptide
  Xaa (11)..(12): Asp-Ser pseudoproline dipeptide
TFXXDYSKYLXXRRAQDFVQWLMNT

SEQ ID NO: 9 - protein, 25 aa, Artificial Sequence
  Synthetic Sequence - C-terminal peptide with pseudoproline pFT
  Xaa (2)..(3): Phe-Thr pseudoproline dipeptide
TXXSDYSKYLDSRRAQDFVQWLMNT

SEQ ID NO: 10 - protein, 25 aa, Artificial Sequence
  Synthetic Sequence - C-terminal peptide with pseudoproline pFT and pDS
  Xaa (2)..(3): Phe-Thr pseudoproline dipeptide
  Xaa (11)..(12): Asp-Ser pseudoproline dipeptide
TXXSDYSKYLXXRRAQDFVQWLMNT

SEQ ID NO: 11 - protein, 29 aa, Artificial Sequence
  Synthetic Sequence - glucagon sequence with pDS
  Xaa (15)..(16): Asp-Ser pseudoproline dipeptide
HSQGTFTSDYSKYLXXRRAQDFVQWLMNT

SEQ ID NO: 12 - protein, 29 aa, Artificial Sequence
  Synthetic Sequence - glucagon sequence with pYS
  Xaa (10)..(11): Tyr-Ser pseudoproline dipeptide
HSQGTFTSDXXKYLDSRRAQDFVQWLMNT

SEQ ID NO: 13 - protein, 29 aa, Artificial Sequence
  Synthetic Sequence - glucagon sequence with pYS and pDS
  Xaa (10)..(11): Tyr-Ser pseudoproline dipeptide
  Xaa (15)..(16): Asp-Ser pseudoproline dipeptide
HSQGTFTSDXXKYLXXRRAQDFVQWLMNT

SEQ ID NO: 14 - protein, 29 aa, Artificial Sequence
  Synthetic Sequence - glucagon sequence with pTS
  Xaa (7)..(8): Thr-Ser pseudoproline dipeptide
HSQGTFXXDYSKYLDSRRAQDFVQWLMNT

SEQ ID NO: 15 - protein, 29 aa, Artificial Sequence
  Synthetic Sequence - glucagon sequence with pTS and pDS
  Xaa (7)..(8): Thr-Ser pseudoproline dipeptide
  Xaa (15)..(16): Asp-Ser pseudoproline dipeptide
HSQGTFXXDYSKYLXXRRAQDFVQWLMNT

SEQ ID NO: 16 - protein, 29 aa, Artificial Sequence
  Synthetic Sequence - glucagon sequence with pFT
  Xaa (6)..(7): Phe-Thr pseudoproline dipeptide
HSQGTXXSDYSKYLDSRRAQDFVQWLMNT

SEQ ID NO: 17 - protein, 29 aa, Artificial Sequence
  Synthetic Sequence - glucagon sequence with pFT and pDS
  Xaa (6)..(7): Phe-Thr pseudoproline dipeptide
  Xaa (15)..(16): Asp-Ser pseudoproline dipeptide
HSQGTXXSDYSKYLXXRRAQDFVQWLMNT

(19) United States
(12) Patent Application Publication
(10) Pub. No.: US 2022/0324936 A1
(43) Pub. Date: Oct. 13, 2022

(54) PROCESS FOR THE MANUFACTURE OF GLUCAGON

(71) Applicant: FRESENIUS KABI IPSUM S.R.L., Cassina de' Pecchi - MILANO (IT)

(72) Inventors: Andrea ORLANDIN, Cassina de' Pecchi - MILANO (IT); Antonio RICCI, Cassina de' Pecchi - MILANO (IT); Walter CABRI, Cassina de' Pecchi - MILANO (IT)

(73) Assignee: FRESENIUS KABI IPSUM S.R.L., Cassina de' Pecchi - MILANO (IT)

(21) Appl. No.: 17/620,433

(22) PCT Filed: Jun. 18, 2020
(86) PCT No.: PCT/EP2020/066910

§ 371 Date: Dec. 17, 2021

(30) Foreign Application Priority Data
Jun. 18, 2019 (EP) 19180875.7

(51) Int. Cl. C07K 14/605

(52) U.S. Cl. CPC C07K 14/605

(57) ABSTRACT

The present invention provides an improved process for the preparation of glucagon, comprising the coupling of an N-terminal tetramer fragment with a C-terminal peptide, comprising at least one pseudoproline. The process is very efficient in avoiding aggregation and obtaining the desired product in high yield and purity.

### FIELD OF THE INVENTION

The present invention provides an improved process for the preparation of high purity glucagon and related intermediates.

### BACKGROUND OF THE INVENTION

Glucagon is a polypeptide hormone, secreted by the α-cells of the pancreatic islets of Langerhans. Glucagon is a single chain peptide consisting of 29 natural amino acids (SEQ ID NO:1, glucagon 1-29) and is represented by the chemical structure shown below:

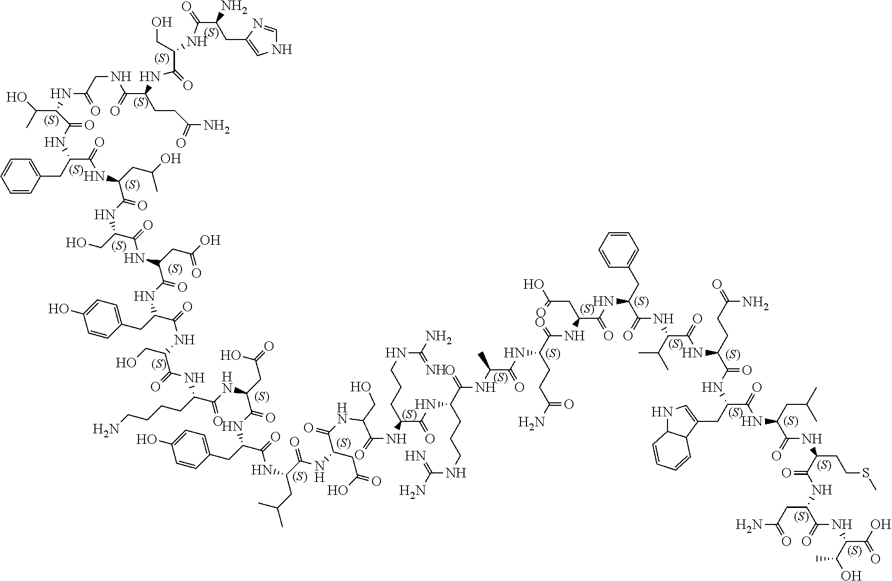

Glucagon was first discovered in 1923 by the chemists Kimball and Murlin in the pancreatic extract. Glucagon is indicated for the treatment of severe hypoglycemic reactions which may occur in the management of insulin treated patients or patients with diabetes mellitus.

Earliest isolation of glucagon was from the pancreatic extracts. The extraction from pancreas is difficult and the product is largely contaminated with insulin. The process produces low yield and therefore large amount of pancreas are required. Moreover, the glucagon of animal origin may induce allergic reaction in some patients making it unfit for use in such cases.

Currently glucagon is produced by recombinant DNA technology or by using Solid Phase Peptide Synthesis (SPPS). Several patents such as U.S. Pat. No. 4,826,763 or 6,110,703 describe the synthesis of glucagon using recombinant DNA technology or genetically modified yeast cells.

Recombinant technology, besides being extremely expensive is also an industrially complicated process. It requires the use of specialised equipment, modified organisms during synthesis and elaborate analytical and purification procedures. Apart from the high cost, the biotechnology processes to produce bio-molecules also suffers from low reproducibility.

The solid phase peptide synthesis process for glucagon is relatively difficult as the long peptide chains often suffer from on-resin aggregation phenomena due to inter- and intra-molecular hydrogen bonding which leads to several truncated sequences appearing as impurities, reducing both the yield and purity of the final compound.

The US patent U.S. Pat. No. 3,642,763 describes the synthesis of glucagon by condensation of an [aa 1-6] and an [aa 7-29] peptide fragment in the presence of N-hydroxy-succinimide or N-hydroxyphthalimide and subsequent splitting of protecting groups in the presence of trifluoroacetic acid. The patent does not disclose the purity of the compound obtained in such a process.

The Chinese patent CN103333239 describes a process for the solid phase peptide synthesis of glucagon wherein the condensation of amino acids is carried out at higher temperatures and wherein the use of pseudoproline dipeptides as protecting groups at position 4/5 and position 7/8, is disclosed. However, the purity of the glucagon obtained via the described process is consistently low.

Therefore, there exists a need for an improved process for the synthesis of glucagon which provides the product in high yield and purity and which is also cost effective and industrially viable.

### OBJECT OF THE INVENTION

It is an objective of the present invention to overcome the above-mentioned drawbacks of the prior art.

It is another objective of the present invention to provide an improved process for the preparation of glucagon, which provides product in high yield as well as high purity.

It is a further objective of the present invention to provide useful intermediates for the synthesis of glucagon.

### SUMMARY OF THE INVENTION

The present invention provides an improved process for the preparation of glucagon.

In one embodiment, the invention relates to a process for the preparation of glucagon comprising the coupling of a N-terminal tetrapeptide (1-4) (SEQ ID NO:2) with a C-terminal peptide (5-29) (SEQ ID NO:3), wherein the C-terminal peptide comprises at least one pseudoproline dipeptide.

The sequence of the N-terminal tetrapeptide (1-4) is His(P)-Ser(P)-Gln(P)-Gly-OH wherein P is a side-chain protecting group or is absent.

The C-terminal peptide (5-29) has the following amino acid sequence Thr(P)-Phe-Thr(P)-Ser(P)-Asp(P)-Tyr(P)-Ser(P)-Lys(P)-Tyr(P)-Leu-Asp(P)-Ser(P)-Arg(P)-Arg(P)-Ala-Gln(P)-Asp(P)-Phe-Val-Gln(P)-Trp(P)-Leu-Met-Asn(P)-Thr(P), which is further specified by the presence of at least one serine or threonine residue which has been reversibly protected as a proline-like acid-labile oxazolidine, also known as pseudoproline; and wherein P is a side-chain protecting group or is absent.

The process according to the invention may be described as a process for the preparation of glucagon comprising the coupling of an N-terminal tetrapeptide (1-4) of glucagon with the above mentioned C-terminal peptide (5-29) of glucagon, wherein at least one serine or threonine in the C-terminal peptide is protected by the use a pseudoproline dipeptide. In a preferred embodiment, the process for the preparation of glucagon comprises the preparation of the C-terminal peptide (5-29), comprising the steps of:
a) coupling an alpha-amino-protected threonine to a resin;
b) selectively cleaving the terminal protecting group;
c) coupling the subsequent alpha-amino-protected amino acid or peptide to the deprotected amino group obtained in step b) in the presence of a coupling reagent;
d) repeating steps b) and c) to elongate the peptide sequence to finally obtain the C-terminal peptide (5-29);

wherein at least one step c) comprises coupling with a pseudoproline dipeptide.

By coupling with a pseudoproline dipeptide the peptide chain is extended by two residues in one step.

A further embodiment of the invention are the different pseudoproline dipeptides and their use in the synthesis of glucagon. The pseudoproline dipeptides are preferably selected from the group consisting of:
Fmoc-Asp(OtBu)-Ser[psi(Me, Me)pro]-OH
Fmoc-Asn(Trt)-Thr[psi(Me, Me)pro]-OH
Fmoc-Tyr(tBu)-Ser[psi(Me, Me)pro]-OH
Fmoc-Phe-Thr[psi(Me, Me)pro]-OH and
Fmoc-Thr(tBu)-Ser[psi(Me, Me)pro]-OH.

More preferably, the process of present invention provides a preparation of glucagon comprising a step of coupling an N-terminal tetrapeptide Boc-His(Trt)-Ser(tBu)-Gln(Trt)-Gly-OH (2) and a C-terminal peptide (5-29), wherein the C-terminal peptide comprises the pseudoproline dipeptide Asp(OtBu)-Ser[psi(Me, Me)pro].

A further embodiment of the present invention relates to C-terminal peptides (5-29) and protected glucagon sequences which are intermediates in the preparation of glucagon.

### DETAILED DESCRIPTION OF THE INVENTION

The present invention relates to a process for the preparation of glucagon of formula I:

His¹-Ser-Gln-Gly-Thr⁵-Phe-Thr-Ser-Asp-Tyr¹⁰-Ser-Lys-Tyr-Leu-Asp¹⁵-Ser-Arg-Arg-Ala-Gln²⁰-Asp-Phe-Val-Gln-Trp²⁵-Leu-Met-Asn-Thr²⁹ (I)

also indicated by the following sequence of amino acids one-letter codes:

HSQGTFTSDYSKYLDSRRAQDFVQWLMNT.

In the synthesis of large peptide molecules, such as glucagon, the conformation of the growing peptide chain and its physico-chemical properties are of considerable importance. The formation of secondary structures often leads to problems of aggregation causing incomplete coupling reactions, resulting in a decrease in the synthetic yield and purity of the final compound.

For instance, it was found that in a stepwise SPPS preparation of glucagon, after the insertion of Gly4 residue (i.e. glycine in position 4), the coupling efficiency dramatically decreases and an efficient completion of glucagon sequence is hampered. This was demonstrated by the presence of the truncated sequences at the residues Gly4, Gln3 and Ser2 in the crude glucagon (after cleavage from resin) and by its very low HPLC purity (see Example 2, Lot 1A of Experimental Part).

Similarly, intra- and inter-molecular aggregation phenomena may be responsible for a decrease in the efficiency of coupling reactions in the synthesis of glucagon even at an earlier stage in the stepwise elongation, for instance after the insertion of Leu14. To solve this problem, it was found that the use of a pseudoproline dipeptide allows to maintain coupling efficiency during the synthesis of the C-terminal peptide (5-29) of glucagon.

Still, the use of pseudoproline dipeptides is not sufficient to obtain crude glucagon in decent yield (see Example 2, Lot 1B of Experimental Part).

It was found that the insertion of the last four amino acids (1-4) of glucagon sequence in one step, through a fragment-based synthesis approach involving coupling of tetrapeptide His-Ser-Gln-Gly, instead of coupling Fmoc-Gly-OH, provides a protected glucagon sequence with surprisingly high purity.

On one side, the use of at least one pseudoproline dipeptide allows an efficient preparation of C-terminal peptide (5-29) of glucagon. On the other, the coupling of glucagon N-terminal tetrapeptide (1-4) with C-terminal peptide (5-29) is very efficient and finally results in a crude product with good yield and high purity.

Therefore, the present invention provides a process for the preparation of glucagon comprising the coupling of an optionally protected tetrapeptide (1-4) of glucagon with a C-terminal peptide (5-29) of glucagon, wherein the C-terminal peptide comprises at least one pseudoproline dipeptide.

The process of the present invention may be performed by SPPS or by LPPS (Liquid Phase Peptide Synthesis) or by mixed SPPS/LPPS techniques, by adapting conditions and methods herein described according to well known practice to the person skilled in the art.

The amino acids employed in the process of the present invention have the natural L-configuration; in general, such amino acids and pseudoproline dipeptides (preferably bearing a terminal protecting group) employed in the process of the present invention are commercially available.

The term “terminal protecting group” as used herein refers to the protecting group for the alpha-amino group of the amino acids or of the peptides used in the preparation of glucagon, or of the complete glucagon sequence, which is cleaved either prior to the coupling to elongate the peptide sequence or at the end of the peptide elongation. Preferably, the terminal protecting group is 9-fluorenylmethyloxycarbonyl (Fmoc) or tert-butyloxycarbonyl (Boc).

The term “resin” is used to describe a functionalized polymeric solid support suitable to perform peptide synthesis. Preferably, the resin in the present context may be selected from the group comprising 2-chlorotrityl chloride (CTC), trityl chloride, Wang, Rink amide, Rink amide AM and Rink amide MBHA resins.

“On-resin aggregation” refers to the secondary structure formation or clumping of the peptide chain due to intra- and intermolecular hydrogen bonding interactions which decrease the availability of the peptide to coupling reaction and hinder the further growth of the peptide chain.

The term “pseudoproline” refers to an oxazolidine as simultaneous protection of the alpha-amino group and the side-chain hydroxy group of serine or threonine via cyclization with an aldehyde or ketone, resulting in oxazolidines exhibiting structural features similar to a proline, (see also T. Haack, M. Mutter, Tetrahedron Lett. 1992, 33, 1589-1592). The pseudoproline dipeptide structure is depicted below, wherein also the position of the Fmoc terminal protecting group is indicated:

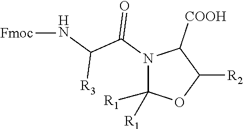

wherein R₁ is hydrogen or methyl; R₂ is hydrogen for Ser and methyl for Thr; and R₃ is the side-chain of the amino acid next to the pseudoproline protected amino acid (configurations at stereocenters are not indicated).

The above pseudoproline dipeptides are also indicated as Fmoc-A₁-A₂[psi(R1,R1)pro]-OH or more simply as pA₁A₂, wherein A₁ and A₂ is either the three-letter or the one-letter code of the involved amino acid, and wherein, in the context of present invention, A₁ refers to aspartic acid, asparagine, tyrosine, phenylalanine or threonine and A₂ refers to serine or threonine. In particular, the abbreviation pA₁A₂ is used throughout the present disclosure when the pseudoproline dipeptide is incorporated into a peptide sequence, i.e. when it is without the terminal group and the free carboxylic acid at the C-terminal end.

The introduction of pseudoprolines dipeptides, for instance Fmoc-protected, into a peptide sequence can be performed in the solid-phase under standard coupling conditions. Once the completed peptide is cleaved from the resin by acidolysis, the pseudoproline is also hydrolysed in the same step, providing the two corresponding native amino acids in the sequence. The cleavage of the pseudoproline protection after completion of the peptide elongation occurs by acid treatment, for instance with a mixture comprising TFA.

As used herein, a “side-chain protecting group” is a protecting group for an amino acid side-chain chemical function which is not removed when the terminal protecting group is removed and is stable during coupling reactions. Preferably, side-chain protecting groups are included to protect side-chains of amino acids which are particularly reactive or labile, to avoid side reactions and/or branching of the growing molecule. Illustrative examples include acid-labile protecting groups, as for instance tert-butyloxycarbonyl (Boc), alkyl groups such as tert-butyl (tBu), trityl (Trt), 2,2,4,6,7-pentamethyldihydrobenzofuran-5-sulfonyl (Pbf) and the like. Other protecting groups may be efficiently used as it is apparent to the person skilled in the art.

The criterion for selecting side-chain protecting groups is that generally the protecting group must be stable to the reaction conditions selected for removing the terminal protecting group at each step of the synthesis and has to be removable upon completion of the synthesis of the desired amino acid sequence under reaction conditions that will not alter the peptide chain.

The term “C-terminal peptide” in the context of present invention refers to a peptide of 25 amino acids in length, sharing the C-terminal amino acid sequence of glucagon ending with a C-terminal threonine (Thr29). This is referred to as SEQ ID NO:3. The C-terminal peptide may be attached to a resin by its C-terminal end, when glucagon is prepared according to the present invention and by SPPS. It is further defined by having an alpha-amino group capable of reacting with the carboxy group of another amino acid, or peptide, at the N-terminal end.

The C-terminal peptide used according to the invention additionally comprises at least one pseudoproline moiety. Such moiety is introduced by way of pseudoproline dipeptides, which are used in the peptide elongation process.

In a preferred embodiment, the process for the preparation of glucagon comprises the preparation of the C-terminal peptide, comprising said at least one pseudoproline moiety.

Another embodiment of the invention relates to the pseudoproline dipeptides and their use in the synthesis of glucagon according to the present invention.

The process for the preparation of glucagon according to the present invention is therefore characterized by the use of one or more of different pseudoproline dipeptides, which may be selected from the group consisting of:
Fmoc-Asp(P)-Ser[psi(R₁, R₁)pro]-OH (Fmoc-pDS)
Fmoc-Asn(P)-Thr[psi(R₁, R₁)pro]-OH (Fmoc-pNT)
Fmoc-Tyr(P)-Ser[psi(R₁, R₁)pro]-OH (Fmoc-pYS)
Fmoc-Phe-Thr[psi(R₁, R₁)pro]-OH (Fmoc-pFT) and
Fmoc-Thr(P)-Ser[psi(R₁, R₁)pro]-OH (Fmoc-pTS),

wherein P is a side-chain protecting group or is absent, and R₁ is hydrogen or methyl (Me). Preferably, the pseudoproline dipeptides are selected from the group consisting of:
Fmoc-Asp(OtBu)-Ser[psi(Me, Me)pro]-OH
Fmoc-Asn(Trt)-Thr[psi(Me, Me)pro]-OH
Fmoc-Tyr(tBu)-Ser[psi(Me, Me)pro]-OH
Fmoc-Phe-Thr[psi(Me, Me)pro]-OH and
Fmoc-Thr(tBu)-Ser[psi(Me, Me)pro]-OH.

A preferred embodiment of present invention is the use of Fmoc-Asp(OtBu)-Ser[psi(Me, Me)pro]-OH in the preparation of glucagon according to the present process. In particular, the introduction of the pseudoproline dipeptide Asp(OtBu)-Ser[psi(Me, Me)pro] in substitution of the residues Asp-Ser in position 15-16 in the C-terminal peptide allowed to maintain the peptide elongation effective until the insertion of Thr5 residue.

The present invention therefore provides a process for the preparation of glucagon comprising the preparation of the C-terminal peptide according to the above defined steps a), b), c) and d), wherein at least one step c) comprises coupling with a pseudoproline dipeptide pDS, preferably with Fmoc-Asp(OtBu)-Ser[psi(Me, Me)pro]-OH for positions 15-16 according to the glucagon sequence.

Further embodiments of the present invention are the C-terminal peptide (5-29) of glucagon and its use in the process for the preparation of glucagon.

The C-terminal peptide comprises at least one pseudoproline dipeptide pA₁A₂ and may be selected from the group comprising:

(SEQ ID NO: 4)
Thr(P)-Phe-Thr(P)-Ser(P)-Asp(P)-Tyr(P)-Ser(P)-
Lys(P)-Tyr(P)-Leu-pDS-Arg(P)-Arg(P)-Ala-Gln(P)-
Asp(P)-Phe-Val-Gln(P)-Trp(P)-Leu-Met-Asn(P)-
Thr(P),
(SEQ ID NO: 5)
Thr(P)-Phe-Thr(P)-Ser(P)-Asp(P)-pYS-Lys(P)-
Tyr(P)-Leu-Asp(P)-Ser(P)-Arg(P)-Arg(P)-Ala-
Gln(P)-Asp(P)-Phe-Val-Gln(P)-Trp(P)-Leu-Met-
Asn(P)-Thr(P),
(SEQ ID NO: 6)
Thr(P)-Phe-Thr(P)-Ser(P)-Asp(P)-pYS-Lys(P)-
Tyr(P)-Leu-pDS-Arg(P)-Arg(P)-Ala-Gln(P)-
Asp(P)-Phe-Val-Gln(P)-Trp(P)-Leu-Met-Asn(P)-
Thr(P),
(SEQ ID NO: 7)
Thr(P)-Phe-pTS-Asp(P)-Tyr(P)-Ser(P)-Lys(P)-
Tyr(P)-Leu-Asp(P)-Ser(P)-Arg(P)-Arg(P)-Ala-
Gln(P)-Asp(P)-Phe-Val-Gln(P)-Trp(P)-Leu-Met-
Asn(P)-Thr(P),
(SEQ ID NO: 8)
Thr(P)-Phe-pTS-Asp(P)-Tyr(P)-Ser(P)-Lys(P)-
Tyr(P)-Leu-pDS-Arg(P)-Arg(P)-Ala-Gln(P)-
Asp(P)-Phe-Val-Gln(P)-Trp(P)-Leu-Met-Asn(P)-
Thr(P),
(SEQ ID NO: 9)
Thr(P)-pFT-Ser(P)-Asp(P)-Tyr(P)-Ser(P)-Lys(P)-
Tyr(P)-Leu-Asp(P)-Ser(P)-Arg(P)-Arg(P)-Ala-
Gln(P)-Asp(P)-Phe-Val-Gln(P)-Trp(P)-Leu-Met-
Asn(P)-Thr(P),
(SEQ ID NO: 10)
Thr(P)-pFT-Ser(P)-Asp(P)-Tyr(P)-Ser(P)-Lys(P)-
Tyr(P)-Leu-pDS-Arg(P)-Arg(P)-Ala-Gln(P)-Asp(P)-
Phe-Val-Gln(P)-Trp(P)-Leu-Met-Asn(P)-Thr(P),

and

wherein P is a side-chain protecting group or is absent, and pA₁A₂ is a pseudoproline dipeptide as defined above.

The C-terminal peptide (5-29) when not carrying a pseudoproline dipeptide as protective unit is generically indicated as SEQ ID NO:3, while SEQ ID NO:4 to SEQ ID NO:10 are specific examples comprising specific pseudoprolines at specified positions hereabove.

In one embodiment, the above optionally protected C-terminal peptides (5-29) of glucagon are attached to a solid support at their C-terminal end, preferably to a Wang resin.

In another embodiment, the above optionally protected C-terminal peptides (5-29) of glucagon are protected also with a terminal protecting group, preferably with Fmoc.

Preferably, the C-terminal peptide (5-29) for the preparation of glucagon according to the present invention is:

(SEQ ID NO: 4)
Thr(P)-Phe-Thr(P)-Ser(P)-Asp(P)-Tyr(P)-Ser(P)-
Lys(P)-Tyr(P)-Leu-pDS-Arg(P)-Arg(P)-Ala-
Gln(P)-Asp(P)-Phe-Val-Gln(P)-Trp(P)-Leu-Met-
Asn(P)-Thr(P),

wherein P and pDS are defined above.

Most preferably the C-terminal peptide for the preparation of glucagon is

Thr(tBu)-Phe-Thr(tBu)-Ser(tBu)-Asp(tBu)-
Tyr(tBu)-Ser(tBu)-Lys(Boc)-Tyr(tBu)-Leu-
pDS-Arg(Pbf)-Arg(Pbf)-Ala-Gln(Trt)-
Asp(tBu)-Phe-Val-Gln(Trt)-Trp(Boc)-Leu-
Met-Asn(Trt)-Thr(tBu), (4a)

wherein pDS is Asp(OtBu)-Ser[psi(Me, Me)pro].

A further aspect of the present invention relates to the N-terminal tetramer peptide (1-4) (or tetrapeptide) which is used in the synthesis of glucagon according to the present invention in the coupling with the C-terminal peptide (5-29) of glucagon, namely:

| | |
|---|---|
| His(P)-Ser(P)-Gln(P)-Gly-OH | (SEQ ID NO: 2) |

wherein P is a side-chain protecting group or is absent.

The above tetramer peptide is preferably protected at the alpha-amino group (of histidine) with a terminal protecting group. Preferably, the terminal protecting group is of carbamate type as, for instance, 9-fluorenylmethyloxycarbonyl (Fmoc) or t-Butyloxycarbonyl (Boc).

More preferably, the terminal protecting group of the tetramer peptide is Boc. In a preferred embodiment, the tetramer peptide used in the process of the present invention is Boc-His(Trt)-Ser(tBu)-Gln(Trt)-Gly-OH (2a).

The N-terminal peptide (1-4) is generically indicated as SEQ ID NO:2, while 2a is a specific example comprising specified protecting groups.

Preferably, the C-terminal peptide and the N-terminal tetramer peptide are prepared using SPPS by stepwise coupling of amino acids, or peptides, according to the required sequence to the C-terminal end amino acid attached to a resin, using at least one of a coupling reagent and an additive.

To prepare the tetramer peptide, preferably a CTC resin is used; to prepare the C-terminal peptide, preferably a Wang resin is used.

The resin is activated by the removal of a protecting group. The activated resin is coupled with the first amino acid, i.e. with Thr29 or with Gly4, wherein the amino acid is protected by a terminal protecting group and optionally a side-chain protecting group.

The terminal protecting group is cleaved in suitable conditions depending on its type.

When the Fmoc group is used, it can be removed by treatment in basic conditions. The base used may be an inorganic or organic base. Preferably the base is an organic base selected from the group comprising piperidine, pyrrolidine, piperazine, tert-butylamine, DBU and diethylamine, preferably piperidine.

When the Boc group is used, it can be removed by treatment in acidic conditions. The acid may be an inorganic or organic acid, as well known to any person skilled in the art. Preferably, the acid is TFA at a suitable concentration.

The coupling of amino acids takes place in the presence of a coupling reagent. The coupling reagent may be selected, among others, from the group comprising N,N′-diisopropylcarbodiimide (DIC), N,N′-dicyclohexylcarbodiimide (DCC), (Benzotriazol-1-yloxy)tripyrrolidinophosphonium hexafluorophosphate (PyBOP), N,N,N′,N′-Tetramethyl-O-(benzotriazol-1-yl)uronium tetrafluoroborate (TBTU), 2-(7-Aza-1H-benzotriazole-1-yl)-1,1,3,3-tetramethyluronium hexafluorophosphate (HATU), 2-(1H-benzotriazole-1-yl)-1,1,3,3-tetramethyluronium hexafluorophosphate (HBTU) and ethyl-dimethylaminopropyl carbodiimide (EDC) etc. Preferably, the reaction is carried out in the presence N,N′-diisopropylcarbodiimide.

In a preferred aspect of present invention, the coupling steps are performed also in the presence of an additive. The presence of an additive, when used in the coupling reaction, reduces loss of configuration at the carboxylic acid residue, increases coupling rates and reduces the risk of racemization. The additive may be selected from the group comprising 1-hydroxybenzotriazole (HOBt), 2-hydroxypyridine N-oxide, N-hydroxysuccinimide, 1-hydroxy-7-azabenzotriazole (HOAt), endo-N-hydroxy-5-norbornene-2,3-dicarboxamide and ethyl 2-cyano-2-hydroxyimino-acetate (OxymaPure), 5-(Hydroxyimino)1,3-dimethylpyrimidine-2,4,6-(1H,3H,5H)-trione (Oxyma B). Preferably, the coupling reaction is carried out in the presence of ethyl 2-cyano-2-hydroxyimino-acetate or of 5-(Hydroxyimino)1,3-dimethylpyrimidine-2,4,6-(1H,3H,5H)-trione.

The coupling reaction may be carried out in the presence of a base selected from the group of tertiary amines comprising diisopropylethylamine (DIEA), triethylamine, N-methylmorpholine, N-methylpiperidine etc; preferably, the reaction is carried out in the presence of DIEA.

The coupling reaction, either involving peptides or amino acids, takes place in the presence of a solvent selected from the group comprising dimethylformamide, dimethylacetamide, dimethylsulfoxide, dichloromethane, chloroform, tetrahydrofuran, 2-methyl tetrahydrofuran and N-methyl pyrrolidine.

Additionally, the unreacted sites on the resin are optionally capped, to avoid truncated sequences and to prevent any side reactions, by a short treatment with a large excess of a highly reactive unhindered reagent, which is chosen according to the unreacted sites to be capped, and according to well known peptide synthesis techniques.

Once the desired peptide sequence has been obtained, the N-terminal tetramer needs to be cleaved from the solid support in order to free the carboxylic acid of Gly4 and provide His(P)-Ser(P)-Gln(P)-Gly-OH, optionally protected with a terminal protecting group. Such cleavage is carried out in conditions suitable to the employed solid support and suitable to keep any protections on the peptide sequence, such as the terminal protecting group and any side-chain protecting groups P. For instance, when CTC resin is used, the cleavage is carried out in an acid solution, such as, for instance a 1% TFA DCM solution.

Once the desired peptide sequence has been obtained, the terminal protecting group on the C-terminal peptide is cleaved to free the alpha-amino group in order to make the C-terminal peptide ready for the final coupling with the N-terminal tetramer.

The present invention provides a process for the preparation of glucagon comprising a step of coupling a N-terminal tetrapeptide (1-4) and a C-terminal peptide (5-29), so to obtain a glucagon peptide sequence. With regard to such coupling of the process of the present invention, all the features as above described apply mutatis mutandis. In particular, it is made reference to the coupling reactions conditions, comprising coupling reagents, additives, solvents, protective groups, terminal protecting group cleavage conditions, which are all easily adaptable in a clear manner by the person skilled in the art.

In yet another aspect, the invention therefore relates to various optionally protected glucagon sequences or fragments which are intermediates in the synthesis of glucagon. The peptide sequences may be selected from the group comprising:

(SEQ ID NO: 11)
His(P)-Ser(P)-Gln(P)-Gly-Thr(P)-Phe-Thr(P)-
Ser(P)-Asp(P)-Tyr(P)-Ser(P)-Lys(P)-Tyr(P)-
Leu-pDS-Arg(P)-Arg(P)-Ala-Gln(P)-Asp(P)-
Phe-Val-Gln(P)-Trp(P)-Leu-Met-Asn(P)-
Thr(P),
(SEQ ID NO: 12)
His(P)-Ser(P)-Gln(P)-Gly-Thr(P)-Phe-Thr(P)-
Ser(P)-Asp(P)-pYS-Lys(P)-Tyr(P)-Leu-Asp(P)-
Ser(P)-Arg(P)-Arg(P)-Ala-Gln(P)-Asp(P)-Phe-
Val-Gln(P)-Trp(P)-Leu-Met-Asn(P)-Thr(P),
(SEQ ID NO: 13)
His(P)-Ser(P)-Gln(P)-Gly-Thr(P)-Phe-Thr(P)-
Ser(P)-Asp(P)-pYS-Lys(P)-Tyr(P)-Leu-pDS-
Arg(P)-Arg(P)-Ala-Gln(P)-Asp(P)-Phe-Val-
Gln(P)-Trp(P)-Leu-Met-Asn(P)-Thr(P),
(SEQ ID NO: 14)
His(P)-Ser(P)-Gln(P)-Gly-Thr(P)-Phe-pTS-
Asp(P)-Tyr(P)-Ser(P)-Lys(P)-Tyr(P)-Leu-
Asp(P)-Ser(P)-Arg(P)-Arg(P)-Ala-Gln(P)-
Asp(P)-Phe-Val-Gln(P)-Trp(P)-Leu-Met-
Asn(P)-Thr(P),
(SEQ ID NO: 15)
His(P)-Ser(P)-Gln(P)-Gly-Thr(P)-Phe-pTS-
Asp(P)-Tyr(P)-Ser(P)-Lys(P)-Tyr(P)-Leu-
pDS-Arg(P)-Arg(P)-Ala-Gln(P)-Asp(P)-Phe-
Val-Gln(P)-Trp(P)-Leu-Met-Asn(P)-Thr(P),
(SEQ ID NO: 16)
His(P)-Ser(P)-Gln(P)-Gly-Thr(P)-pFT-
Ser(P)-Asp(P)-Tyr(P)-Ser(P)-Lys(P)-
Tyr(P)-Leu-Asp(P)-Ser(P)-Arg(P)-Arg(P)-
Ala-Gln(P)-Asp(P)-Phe-Val-Gln(P)-Trp(P)-
Leu-Met-Asn(P)-Thr(P),
and
(SEQ ID NO: 17)
His(P)-Ser(P)-Gln(P)-Gly-Thr(P)-pFT-Ser(P)-
Asp(P)-Tyr(P)-Ser(P)-Lys(P)-Tyr(P)-Leu-
pDS-Arg(P)-Arg(P)-Ala-Gln(P)-Asp(P)-Phe-
Val-Gln(P)-Trp(P)-Leu-Met-Asn(P)-Thr(P),,

wherein P is a side-chain protecting group or is absent and pA₁A₂ is a pseudoproline dipeptide as defined above.

The glucagon peptide sequence (1-29) is indicated as SEQ ID NO:1, while the above sequences represented in SEQ ID NOs 11-17 are specific examples of glucagon sequences comprising one or more pseudoproline dipeptides at specified positions as protected moieties.

In one embodiment, the above optionally protected intermediate glucagon sequences are attached to a solid support at their C-terminal end, preferably to a Wang resin.

In another embodiment, the above optionally protected intermediate glucagon sequences are protected also with a terminal protecting group, preferably with Boc.

In a preferred embodiment, the protected glucagon sequence which is intermediate in the synthesis of glucagon is

(SEQ ID NO: 11)
His(P)-Ser(P)-Gln(P)-Gly-Thr(P)-Phe-Thr(P)-
Ser(P)-Asp(P)-Tyr(P)-Ser(P)-Lys(P)-Tyr(P)-
Leu-pDS-Arg(P)-Arg(P)-Ala-Gln(P)-Asp(P)-Phe-
Val-Gln(P)-Trp(P)-Leu-Met-Asn(P)-Thr(P).

In an even more preferred embodiment, the intermediate protected sequence of the process of the present invention for the preparation of glucagon is

Boc-His(Trt)-Ser(tBu)-Gln(Trt)-Gly-Thr(tBu)-
Phe-Thr(tBu)-Ser(tBu)-Asp(tBu)-Tyr(tBu)-
Ser(tBu)-Lys(Boc)-Tyr(tBu)-Leu-pDS-Arg(Pbf)-
Arg(Pbf)-Ala-Gln(Trt)-Asp(tBu)-Phe-Val-
Gln(Trt)-Trp(Boc)-Leu-Met-Asn(Trt)-Thr(tBu)-
Wang resin (11a).

Further deprotection of the protected glucagon sequence provides crude glucagon, which may optionally be purified.

In a preferred embodiment, when SPPS is used, the protected glucagon sequence is finally deprotected and cleaved from the resin, either simultaneously or in two steps, providing crude glucagon, which may optionally be purified.

Deprotection and cleavage conditions generally depend on the nature of the protecting groups and of the resin used: in a preferred embodiment, deprotection and cleavage are performed by treatment with an acid; preferably, with a mixture comprising an acid, for instance trifluoroacetic acid (TFA), or the like. Optionally, the cleavage mixture may comprise one or more scavengers. Scavengers are substances, like, for instance, anisole, thioanisole, triisopropylsilane (TIS), 1,2-ethanedithiol (EDT) and phenol, capable of minimize modification or destruction of the sensitive deprotected side chains, such as those of arginine residues, in the cleavage environment.

For instance, when a Wang resin is used, such cleavage/deprotection step is preferably performed by using a mixture comprising TFA, TIS and EDT, for instance a TFA/TIS/H₂O/EDT/L-Methionine/NH₄I (92.5:2:2:2:1:0.5 v/v/v/v/w/w) mixture. The crude glucagon obtained may be optionally purified by crystallization or chromatographic techniques well known in the art.

The inventors of the present process have found that the use of the above described coupling between a N-terminal tetrapeptide (1-4) and a C-terminal peptide (5-29), as defined above and according to the above described methods, provides glucagon in great yield and high purity, which makes it suitable for large scale industrial production.

### Abbreviations

SPPS Solid phase peptide synthesis

LPPS Liquid phase peptide synthesis

MBHA resin Methyl benzhydryl amide resin

Fmoc 9-fluorenylmethyloxycarbonyl

Boc t-Butyloxycarbonyl

Trt Trityl

tBu Tert-butyl

Pbf 2,2,4,6,7-pentamethyldihydrobenzofuran-5-sulfonyl

HPLC High performance liquid chromatography

h/min hour/minutes

DIEA Diisopropylethylamine

DBU 1,8-Diazabicyclo[5.4.0]undec-7-ene

DMAP 4-DimethylaminopyridineTFA Trifluoroacetic acid

Ac₂O Acetic anhydride

DMF N,N-Dimethylformamide

DCM Dichloromethane

ACN Acetonitrile

MeOH Methanol

DIPE Diisopropylether

TIS Triisopropylsilane

EDT 1,2-ethanedithiol

DIC Diisopropylcarbodiimide

DCC Dicyclohexylcarbodiimide

EDC Ethyl-dimethylaminopropyl carbodiimide

HOBt 1-Hydroxybenzotriazole

HOAt 1-Hydroxy-7-azabenzotriazole

TBTU N,N,N′,N′-Tetramethyl-O-(benzotriazol-1-yl)uronium tetrafluoroborate

HBTU 3-[Bis(dimethylamino)methyliumyl]-3H-benzotriazol-1-oxide hexafluorophosphate

HATU 2-(7-Aza-1H-benzotriazole-1-yl)-1,1,3,3-tetramethyluronium hexafluorophosphate

PyBOP (Benzotriazol-1-yloxy)-tripyrrolidinophosphoniumhexafluorophosphate

Oxyma/OxymaPure Ethyl 2-cyano-2-hydroxyimino-acetate

Oxyma B 5-(Hydroxyimino)1,3-dimethylpyrimidine-2,4,6-(1H,3H,5H)-trione

### EXPERIMENTAL PART

Detailed experimental conditions suitable for the preparation of glucagon according to the present invention are provided by the following examples, which are intended to be illustrative, and not limiting, of all possible embodiments of the invention.

Unless otherwise noted, all materials, solvents and reagents were obtained from commercial suppliers, of the best grade, and used without further purification.

Assays (%) are calculated by HPLC, comparing the peak area of the sample with the peak area of the standard. The molar yields (%) are calculated considering the final moles obtained (based on Assay) divided by the initial moles.

### Example 1. Synthesis of Boc-His(Trt)-Ser(tBu)-Gln(Trt)-Gly-OH

Synthesis of the title tetrapeptide was carried out by SPPS on CTC resin (2 grams). After swelling the resin with DCM (20 mL), Fmoc-Gly-OH (1 eq with respect to the loading of the resin) and DIEA (2 eq) dissolved in DCM (12 mL) were added to the resin and left to react for 1 hour. The resin was then washed with DCM (3×12 mL) and the residual free chloride groups were replaced with MeOH and DIEA in DCM. Residual hydroxyl groups were capped with Ac₂O 0.5 M in DCM (12 mL for 15 min) and washed with DCM (3×12 mL). The resin was than swelled with DMF (12 mL) for 30 minutes. Fmoc-group was removed by treatment with a 20% piperidine in DMF (2×12 mL, 10 minutes per cycle) and washed with DMF (4×12 mL, 2×5 minutes and 2×10 minutes). The loading of the resin after the insertion of the first amino acid was evaluated by UV measurement of the deprotection solution at 301 nm, providing a loading of 1.2 mmol/g.

The next aminoacids used in the peptide elongation were the following (ordered from the first to the last): Fmoc-Gln(Trt)-OH, Fmoc-Ser(tBu)-OH and Boc-His(Trt)-OH.

Except for the Boc-His(Trt)-OH residue, the Fmoc-aminoacid (2 eq with respect to resin loading, in this case 4.8 mmol) was pre-activated with DIC (2 eq) and OxymaPure (2 eq) for 3 minutes, then added to the resin and coupled for 60 minutes. Oxyma B was used in place of OxymaPure for the activation of Boc-His(Trt)-OH. After the termination of the peptide chain the peptidyl resin was washed with DMF (3×12 mL), DCM (3×12 mL) and dried up to constant weight. Full protected peptide was obtained by a treatment with a 1% TFA in DCM solution (10 mL×5; stirred for 15 minutes each time). Cleavage mixtures were pooled, washed with water and precipitated with DIPE (150 ml respect the cleavage mixture volume).

The solid was filtered, washed other 3 times with 20 mL of DIPE and dried in vacuo to get 2.4 g of crude Boc-His(Trt)-Ser(tBu)-Gln(Trt)-Gly-OH (2.2 mmol) with an HPLC purity of 97%. Molar Yield: 91.6%.

### Example 2. Preparation of Glucagon

Loading of Resin

Synthesis of glucagon was carried out by SPPS on Wang resin (3 grams). After swelling the resin with DMF (10 mL), Fmoc-Thr(tBu)-OH (4 eq with respect to the loading of the resin) was pre-activated with DIC and DMAP (2 and 0.1 eq, respectively) for 5 min in DMF (18 mL), then added to the resin and coupled for 60 min. The resin was then washed with DMF (3×6 mL) and the residual free hydroxyl groups were capped with Ac₂O 0.5 M in DMF (6 mL for 15 min) and washed with DMF (3×6 mL). Fmoc group was removed by treatment with 20% piperidine in DMF (2×6 mL, 10 min for cycle) and washed with DMF (4×6 mL, 2×5 min and 2×10 min). The loading of the resin after the insertion of the first amino acid was evaluated by UV measurement of the deprotection solution at 301 nm, providing a loading of 0.7 mmol/g.

The resin thus obtained was split in three portions (1 gram of starting resin each): one was used for the SPPS synthesis of glucagon employing only standard Fmoc-protected aminoacids (Lot 1A), the second one employing the pseudoproline dipeptide residue Fmoc-Asp(OtBu)-Ser[psi(Me,Me)pro]-OH (positions 15-16, Lot 1B), and the third one employing both the pseudoproline dipeptide residue Fmoc-Asp(OtBu)-Ser[psi(Me,Me)pro]-OH and the tetrapeptide Boc-His(Trt)-Ser(tBu)-Gln(Trt)-Gly-OH (Lot 1C).

Lot 1A (Reference)

Preparation was carried out by employing the following amino acids, ordered from the first to the last attached to H-Thr-Wang resin obtained as described above:

Fmoc-Asn(Trt)-OH, Fmoc-Met-OH, Fmoc-Leu-OH, Fmoc-Trp(Boc)-OH, Fmoc-Gln(Trt)-OH, Fmoc-Val-OH, Fmoc-Phe-OH, Fmoc-Asp(tBu)-OH, Fmoc-Gln(Trt)-OH, Fmoc-Ala-OH, Fmoc-Arg(Pbf)-OH, Fmoc-Arg(Pbf)-OH, Fmoc-Ser(tBu)-OH, Fmoc-Asp(tBu)-OH, Fmoc-Leu-OH, Fmoc-Tyr(tBu)-OH, Fmoc-Lys(Boc)-OH, Fmoc-Ser(tBu)-OH, Fmoc-Tyr(tBu)-OH, Fmoc-Asp(tBu)-OH, Fmoc-Ser(tBu)-OH, Fmoc-Thr(tBu)-OH, Fmoc-Phe-OH, Fmoc-Thr(tBu)-OH, Fmoc-Gly-OH, Fmoc-Gln(Trt)-OH, Fmoc-Ser(tBu)-OH, Boc-His(Trt)-OH.

In each step, the Fmoc-protected amino acid (4 eq with respect to resin loading, i.e. 2.8 mmol) was pre-activated with DIC (4 eq) and OxymaPure (4 eq) for 3 min in DMF (6 mL), then added to the resin and coupled for 60 min. After each coupling, the unreacted amino groups were capped using Ac₂O 0.5 M in DMF. Fmoc groups were removed by treatment with 20% piperidine in DMF (2×6 mL, 10 min per cycle) and subsequent washing of the resin with DMF (4×6 mL, 2×5 min and 2×10 min), to allow the insertion of the next amino acid residue. After completion of the peptide sequence, the peptidyl resin was washed with DMF (3×6 mL), DCM (3×6 mL) and dried up to constant weight. Dry peptidyl resin was suspended in 20 mL of a TFA/TIS/H₂O/EDT/Methionine/NH₄I (92.5:2:2:2:1:0.5 v/v/v/v/w/w) mixture, pre-cooled to 0-5° C. and stirred for 4 h at room temperature. The resin was filtered off and cold diisopropylether (80 mL) was added to the solution. The obtained pale yellow suspension was stirred at 0-5° C. The solid was filtered, washed further 3 times with 20 mL of diisopropylether and dried in vacuo to get 2.4 g of crude glucagon (0.10 mmol, assay 15%) with an HPLC purity of 37%. Molar Yield: 15%.

Lot 1B (Reference)

Preparation was carried out by employing the following amino acids and peptides, ordered from the first to the last attached to H-Thr-Wang resin obtained as described above:

Fmoc-Asn(Trt)-OH, Fmoc-Met-OH, Fmoc-Leu-OH, Fmoc-Trp(Boc)-OH, Fmoc-Gln(Trt)-OH, Fmoc-Val-OH, Fmoc-Phe-OH, Fmoc-Asp(tBu)-OH, Fmoc-Gln(Trt)-OH, Fmoc-Ala-OH, Fmoc-Arg(Pbf)-OH, Fmoc-Arg (Pbf)-OH, Fmoc-Asp(OtBu)-Ser[psi(Me,Me)pro]-OH, Fmoc-Leu-OH, Fmoc-Tyr(tBu)-OH, Fmoc-Lys(Boc)-OH, Fmoc-Ser(tBu)-OH, Fmoc-Tyr(tBu)-OH, Fmoc-Asp(tBu)-OH, Fmoc-Ser(tBu)-OH, Fmoc-Thr(tBu)-OH, Fmoc-Phe-OH, Fmoc-Thr(tBu)-OH, Fmoc-Gly-OH, Fmoc-Gln(Trt)-OH, Fmoc-Ser(tBu)-OH, Boc-His(Trt)-OH.

In each step, the Fmoc-protected amino acid (4 eq with respect to resin loading, i.e. 2.8 mmol) was pre-activated with DIC (4 eq) and OxymaPure (4 eq) for 3 min in DMF (6 mL), then added to the resin and coupled for 60 min. Pseudoproline residue Fmoc-Asp(OtBu)-Ser[psi(Me,Me)pro]-OH (3 eq) was coupled after pre-activation with DIC and OxymaPure (3 eq) for 3 min in DMF (6 mL), then added to the resin and coupled for 90 min. After each coupling, the unreacted amino groups were capped using Ac₂O 0.5 M in DMF. Fmoc groups were removed by treatment with 20% piperidine in DMF (2×6 mL, 10 min per cycle) and subsequent washing of the resin with DMF (4×6 mL, 2×5 min and 2×10 min), to allow the insertion of the next residue. After completion of the peptide sequence, the peptidyl resin was washed with DMF (3×6 mL), DCM (3×6 mL) and dried up to constant weight. Dry peptidyl resin was suspended in 20 mL of a TFA/TIS/H₂O/EDT/L-Methionine/NH₄I (92.5:2:2:2:1:0.5 v/v/v/v/w/w) mixture, pre-cooled to 0-5° C. and stirred for 4 h at room temperature. The resin was filtered off and cold diisopropylether (80 ml) was added to the solution. The obtained pale yellow suspension was stirred at 0-5° C. The solid was filtered, washed further 3 times with 20 mL of diisopropylether and dried in vacuo to get 1.7 g of crude glucagon (0.02 mmol, assay 4%) with an HPLC purity of 8%. Molar Yield: 3%.

Lot 1C

Preparation was carried out by employing the following amino acids and peptides, ordered from the first to the last attached to H-Thr-Wang resin obtained as described above:

Fmoc-Asn(Trt)-OH, Fmoc-Met-OH, Fmoc-Leu-OH, Fmoc-Trp(Boc)-OH, Fmoc-Gln(Trt)-OH, Fmoc-Val-OH, Fmoc-Phe-OH, Fmoc-Asp(tBu)-OH, Fmoc-Gln(Trt)-OH, Fmoc-Ala-OH, Fmoc-Arg(Pbf)-OH, Fmoc-Arg (Pbf)-OH, Fmoc-Asp(OtBu)-Ser[psi(Me,Me)pro]-OH, Fmoc-Leu-OH, Fmoc-Tyr(tBu)-OH, Fmoc-Lys(Boc)-OH, Fmoc-Ser(tBu)-OH, Fmoc-Tyr(tBu)-OH, Fmoc-Asp(tBu)-OH, Fmoc-Ser(tBu)-OH, Fmoc-Thr(tBu)-OH, Fmoc-Phe-OH, Fmoc-Thr(tBu)-OH, Boc-His(Trt)-Ser(tBu)-Gln(Trt)-Gly-OH.

In each step, the Fmoc-protected amino acid (4 eq with respect to resin loading, i.e. 2.8 mmol) was pre-activated with DIC (4 eq) and OxymaPure (4 eq) for 3 min in DMF (6 mL), then added to the resin and coupled for 60 min. Pseudoproline residue Fmoc-Asp(OtBu)-Ser[psi(Me,Me)pro]-OH (3 eq, i.e. 2.1 mmol) and the tetrapeptide Boc-His(Trt)-Ser(tBu)-Gln(Trt)-Gly-OH (2 eq) were coupled after pre-activation with DIC and OxymaPure (2 eq) for 15 min at 40° C. in DMF (16 mL), then added to the resin and coupled for 180 min. After each coupling, the unreacted amino groups were capped using Ac₂O 0.5 M in DMF. Fmoc groups were removed by treatment with 20% piperidine in DMF (2×6 mL, 10 min per cycle) and washed with DMF (4×6 mL, 2×5 min and 2×10 min), to allow the insertion of the next residue. After completion of the peptide sequence, the peptidyl resin was washed with DMF (3×6 mL), DCM (3×6 mL) and dried up to constant weight. Dry peptidyl resin was suspended in 20 mL of a TFA/TIS/H₂O/EDT/L-Methionine/NH₄I (92.5:2:2:2:1:0.5 v/v/v/v/w/w) mixture, pre-cooled to 0-5° C. and stirred for 4 h at room temperature. The resin was filtered off and cold diisopropylether (80 ml) was added to the solution. The obtained pale yellow suspension was stirred at 0-5° C. The solid was filtered, washed further 3 times with 20 mL of diisopropylether and dried in vacuo to get 2.75 g of crude glucagon (0.40 mmol, assay 50%) with an HPLC purity of 80%. Molar Yield: 58%.

1. A process for the preparation of glucagon of formula I (SEQ ID NO: 1)
His¹-Ser-Gln-Gly-Thr⁵-Phe-Thr-Ser-Asp-Tyr¹⁰-Ser-Lys-Tyr-Leu-Asp¹⁵-Ser-Arg-Arg-Ala-Gln²⁰-Asp-Phe-Val-Gln-Trp²⁵-Leu-Met-Asn-Thr²⁹ (I)
the process comprising the coupling of a N-terminal tetrapeptide (1-4) comprising SEQ ID NO: 2 with a C-terminal peptide (5-29) comprising SEQ ID NO: 3, wherein the C-terminal peptide comprises at least one pseudoproline dipeptide.

2. The process according to claim 1, wherein the N-terminal tetrapeptide (1-4) is His(P)-Ser(P)-Gln(P)-Gly-OH, and the C-terminal peptide (5-29) is Thr(P)-Phe-Thr(P)-Ser(P)-Asp(P)-Tyr(P)-Ser(P)-Lys(P)-Tyr(P)-Leu-Asp(P)-Ser(P)-Arg(P)-Arg(P)-Ala-Gln(P)-Asp(P)-Phe-Val-Gln(P)-Trp(P)-Leu-Met-Asn(P)-Thr(P), and wherein at least one serine or threonine residue is protected by a pseudoproline; and wherein P is a side-chain protecting group or is absent.

3. The process according to claim 1, wherein the process is performed by solid phase peptide synthesis (SPPS).

4. The process according to claim 1, further comprising preparing the C-terminal peptide (5-29) by a method comprising the steps of:
a) coupling an alpha-amino-protected threonine to a resin;
b) selectively cleaving the terminal protecting group;
c) coupling the subsequent alpha-amino-protected amino acid or peptide to the deprotected amino group obtained in step b) in the presence of a coupling reagent; and
d) repeating steps b) and c) to elongate the peptide sequence;
wherein at least one step c) comprises coupling with a pseudoproline dipeptide.

5. The process according to claim 1, wherein the pseudoproline dipeptide is:
Fmoc-Asp(P)-Ser[psi(R₁, R₁)pro]-OH (Fmoc-pDS)
Fmoc-Asn(P)-Thr[psi(R₁, R₁)pro]-OH (Fmoc-pNT)
Fmoc-Tyr(P)-Ser[psi(R₁, R₁)pro]-OH (Fmoc-pYS)
Fmoc-Phe-Thr[psi(R₁, R₁)pro]-OH (Fmoc-pFT) or
Fmoc-Thr(P)-Ser[psi(R₁, R₁)pro]-OH (Fmoc-pTS),
wherein P is a protecting group or is absent, and R₁ is hydrogen or methyl.

6. The process according to claim 5, wherein the pseudoproline dipeptide is:
Fmoc-Asp(OtBu)-Ser[psi(Me, Me)pro]-OH
Fmoc-Asn(Trt)-Thr[psi(Me, Me)pro]-OH
Fmoc-Tyr(tBu)-Ser[psi(Me, Me)pro]-OH
Fmoc-Phe-Thr[psi(Me, Me)pro]-OH or
Fmoc-Thr(tBu)-Ser[psi(Me, Me)pro]-OH.

7. The process according to claim 1, wherein the C-terminal peptide is:
(SEQ ID NO: 4)
Thr(P)-Phe-Thr(P)-Ser(P)-Asp(P)-Tyr(P)-Ser(P)-
Lys(P)-Tyr(P)-Leu-pDS-Arg(P)-Arg(P)-Ala-
Gln(P)-Asp(P)-Phe-Val-Gln(P)-Trp(P)-Leu-Met-
Asn(P)-Thr(P),
(SEQ ID NO: 5)
Thr(P)-Phe-Thr(P)-Ser(P)-Asp(P)-pYS-Lys(P)-
Tyr(P)-Leu-Asp(P)-Ser(P)-Arg(P)-Arg(P)-Ala-
Gln(P)-Asp(P)-Phe-Val-Gln(P)-Trp(P)-Leu-Met-
Asn(P)-Thr(P),
(SEQ ID NO: 6)
Thr(P)-Phe-Thr(P)-Ser(P)-Asp(P)-pYS-Lys(P)-
Tyr(P)-Leu-pDS-Arg(P)-Arg(P)-Ala-Gln(P)-
Asp(P)-Phe-Val-Gln(P)-Trp(P)-Leu-Met-Asn(P)-
Thr(P),
(SEQ ID NO: 7)
Thr(P)-Phe-pTS-Asp(P)-Tyr(P)-Ser(P)-Lys(P)-
Tyr(P)-Leu-Asp(P)-Ser(P)-Arg(P)-Arg(P)-Ala-
Gln(P)-Asp(P)-Phe-Val-Gln(P)-Trp(P)-Leu-Met-
Asn(P)-Thr(P),
(SEQ ID NO: 8)
Thr(P)-Phe-pTS-Asp(P)-Tyr(P)-Ser(P)-Lys(P)-
Tyr(P)-Leu-pDS-Arg(P)-Arg(P)-Ala-Gln(P)-
Asp(P)-Phe-Val-Gln(P)-Trp(P)-Leu-Met-Asn(P)-
Thr(P),
(SEQ ID NO: 9)
Thr(P)-pFT-Ser(P)-Asp(P)-Tyr(P)-Ser(P)-
Lys(P)-Tyr(P)-Leu-Asp(P)-Ser(P)-Arg(P)-
Arg(P)-Ala-Gln(P)-Asp(P)-Phe-Val-
Gln(P)-Trp(P)-Leu-Met-Asn(P)-Thr(P),
[[and]] or
(SEQ ID NO: 10)
Thr(P)-pFT-Ser(P)-Asp(P)-Tyr(P)-Ser(P)-
Lys(P)-Tyr(P)-Leu-pDS-Arg(P)-Arg(P)-
Ala-Gln(P)-Asp(P)-Phe-Val-Gln(P)-Trp(P)-
Leu-Met-Asn(P)-Thr(P),
wherein P is a side-chain protecting group or is absent.

8. The process according to claim 7, wherein the C-terminal peptide is
Thr(tBu)-Phe-Thr(tBu)-Ser(tBu)-Asp(tBu)-
Tyr(tBu)-Ser(tBu)-Lys(Boc)-Tyr(tBu)-Leu-
pDS-Arg(Pbf)-Arg(Pbf)-Ala-Gln(Trt)-
Asp(tBu)-Phe-Val-Gln(Trt)-Trp(Boc)-Leu-
Met-Asn(Trt)-Thr(tBu) (4a)
wherein pDS is Asp(OtBu)-Ser[psi(Me, Me)pro].

9. The process according to claim 1, wherein the coupling is performed in the presence of a coupling reagent.

10. The process according to claim 9, wherein the coupling reagent is diisopropylcarbodiimide, dicyclohexylcarbodiimide, (benzotriazol-1-yloxy)tripyrrolidinophosphonium hexafluorophosphate, 2-(7-aza-1H-benzotriazole-1-yl)-1,1,3,3-tetramethyluronium hexafluorophosphate, 2-(1H-benzotriazole-1-yl)-1,1,3,3-tetramethyluronium hexafluorophosphate or ethyl-dimethylaminopropyl carbodiimide.

11. The process according to claim 4, wherein step c) comprises coupling with Fmoc-Asp(OtBu)-Ser[psi(R₁, R₁)pro]-OH, wherein R₁ is hydrogen or methyl.

12. A protected glucagon sequence selected from:
(SEQ ID NO: 11)
His(P)-Ser(P)-Gln(P)-Gly-Thr(P)-Phe-Thr(P)-
Ser(P)-Asp(P)-Tyr(P)-Ser(P)-Lys(P)-Tyr(P)-
Leu-pDS-Arg(P)-Arg(P)-Ala-Gln(P)-Asp(P)-Phe-
Val-Gln(P)-Trp(P)-Leu-Met-Asn(P)-Thr(P),
(SEQ ID NO: 12)
His(P)-Ser(P)-Gln(P)-Gly-Thr(P)-Phe-Thr(P)-
Ser(P)-Asp(P)-pYS-Lys(P)-Tyr(P)-Leu-Asp(P)-
Ser(P)-Arg(P)-Arg(P)-Ala-Gln(P)-Asp(P)-Phe-
Val-Gln(P)-Trp(P)-Leu-Met-Asn(P)-Thr(P),
(SEQ ID NO: 13)
His(P)-Ser(P)-Gln(P)-Gly-Thr(P)-Phe-Thr(P)-
Ser(P)-Asp(P)-pYS-Lys(P)-Tyr(P)-Leu-pDS-
Arg(P)-Arg(P)-Ala-Gln(P)-Asp(P)-Phe-Val-
Gln(P)-Trp(P)-Leu-Met-Asn(P)-Thr(P),
(SEQ ID NO: 14)
His(P)-Ser(P)-Gln(P)-Gly-Thr(P)-Phe-pTS-
Asp(P)-Tyr(P)-Ser(P)-Lys(P)-Tyr(P)-Leu-
Asp(P)-Ser(P)-Arg(P)-Arg(P)-Ala-Gln(P)-
Asp(P)-Phe-Val-Gln(P)-Trp(P)-Leu-Met-
Asn(P)-Thr(P),
(SEQ ID NO: 15)
His(P)-Ser(P)-Gln(P)-Gly-Thr(P)-Phe-pTS-
Asp(P)-Tyr(P)-Ser(P)-Lys(P)-Tyr(P)-Leu-
pDS-Arg(P)-Arg(P)-Ala-Gln(P)-Asp(P)-Phe-
Val-Gln(P)-Trp(P)-Leu-Met-Asn(P)-Thr(P),
(SEQ ID NO: 16)
His(P)-Ser(P)-Gln(P)-Gly-Thr(P)-pFT-
Ser(P)-Asp(P)-Tyr(P)-Ser(P)-Lys(P)-Tyr(P)-
Leu-Asp(P)-Ser(P)-Arg(P)-Arg(P)-Ala-Gln(P)-
Asp(P)-Phe-Val-Gln(P)-Trp(P)-Leu-Met-
Asn(P)-Thr(P),
and
(SEQ ID NO: 17)
His(P)-Ser(P)-Gln(P)-Gly-Thr(P)-pFT-Ser(P)-
Asp(P)-Tyr(P)-Ser(P)-Lys(P)-Tyr(P)-Leu-
pDS-Arg(P)-Arg(P)-Ala-Gln(P)-Asp(P)-Phe-
Val-Gln(P)-Trp(P)-Leu-Met-Asn(P)-Thr(P),
wherein P is a side-chain protecting group or is absent.

13. The protected glucagon sequence according to claim 12, which is
Boc-His(Trt)-Ser(tBu)-Gln(Trt)-Gly-Thr(tBu)-
Phe-Thr(tBu)-Ser(tBu)-Asp(tBu)-Tyr(tBu)-
Ser(tBu)-Lys(Boc)-Tyr(tBu)-Leu-pDS-Arg(Pbf)-
Arg(Pbf)-Ala-Gln(Trt)-Asp(tBu)-Phe-Val-
Gln(Trt)-Trp(Boc)-Leu-Met-Asn(Trt)-Thr(tBu)-
Wang resin (11a),
wherein pDS is Asp(OtBu)-Ser[psi(Me, Me)pro].

14. The process according to claim 9, wherein the coupling is performed in the presence of diisopropylcarbodiimide and ethyl 2-cyano-2-hydroxyimino-acetate.

15. The process according to claim 1, wherein the N-terminal tetrapeptide (1-4) is
Boc-His(Trt)-Ser(tBu)-Gln(Trt)-Gly-OH (2a).

16. A process for the preparation of glucagon, the process comprising coupling Boc-His(Trt)-Ser(tBu)-Gln(Trt)-Gly-OH with a peptide, to produce glucagon.
